(19) United States
(12) Patent Application Publication
(10) Pub. No.: US 2024/0084234 A1
(43) Pub. Date: Mar. 14, 2024

(54) Microfluidic Chips for Neurological and Other Biological Studies

(71) Applicant: University of Cincinnati, Cincinnati, OH (US)

(72) Inventors: Tao Li, Mason, OH (US); Ziyuan Guo, Union, KY (US); Tianshuo Wang, Cincinnati, OH (US)

(73) Assignee: University of Cincinnati, Cincinnati, OH (US)

(21) Appl. No.: 18/272,462

(22) PCT Filed: Jan. 18, 2022
(86) PCT No.: PCT/US22/12834

§ 371 Date: Jul. 14, 2023

Related U.S. Application Data
(60) Provisional application No. 63/138,070, filed on Jan. 15, 2021.

(51) Int. Cl. C12M 3/06; C12M 1/00; C12M 1/34; C12N 5/00; C12N 5/0797

(52) U.S. Cl. CPC C12M 23/16; C12M 23/34; C12M 41/46; C12N 5/0068; C12N 5/0623

(57) ABSTRACT

The present invention involves a compartmentalized microfluidic device using one or more separators. Each separator has a plurality of microfluidic channels and the separators are oriented in a perpendicular direction to the substrate. The vertical integration of the microfluidic components enables realization of 3D device features with high aspect ratio.

### CROSS REFERENCE TO RELATED APPLICATIONS

This application is a continuation of PCT Application No. PCT/US22/12834 filed Jan. 18, 2022, which claims benefit of U.S. Provisional Application Ser. No. 63/138,070, filed Jan. 15, 2021, which applications are hereby incorporated by reference in their entirety.

### TECHNICAL FIELD

The present invention relates generally to microfluidic chips.

### BACKGROUND OF THE INVENTION

A major unresolved hurdle in current study of human cells or organoids-based assays is that in vitro cultures weakly recapitulate the physiology of neuron-to-neuron communication, especially the long-distance projections between neurons of different identities. Previous approaches have provided a proof of concept for microfluidics as a strategy to compartmentalize culture in brain circuit studies, in which planar 2D microchannels were formed typically by molding polydimethylsiloxane (PDMS). Furthermore, in most cases, they are not amenable to high-definition functional studies, e.g. patch clamp or microelectrode arrays (MEAs). Similarly, this situation also applies to neuronal projections between neurons and tissue from other types of organs such as muscle, heart, etc., as well as interactions and/or communications between any other types of biological cells, tissues, or organs/organoids.

Take neuron-to-neuron communication as an example. A neural circuit is a group of neurons interconnected to each other to perform certain function, while interconnected neural circuits form larger brain/neural networks. The study of neural circuits is important to help understand brain functions and the causes of neurological complications. Dysfunctional neural circuits are proven to be responsible for many neurological disorders such as autism spectrum disorder and schizophrenia, while properly triggered circuits can enhance certain brain functions, e.g., the reading circuit in educational scenarios. Traditionally, neural circuit characterizations are done by in vivo magnetic resonance imaging (MRI), which typically can only identify the circuit locations in the brain.

Compartmentalized microfluidic devices can provide an in vitro environment for effective modeling and studying neural circuits, allowing better understanding of how neurons interact with each other by culturing neurons in separate compartments with real time examination of neural activities. Previous works have demonstrated such devices for neural circuit studies, in which planar two-dimensional (2D) configurations were used to implement device features in a horizonal arrangement parallel to the substrate surface with limited height, preventing three-dimensional (3D) implementation of device features away from the substrate. These devices are typically formed by molding polydimethylsiloxane (PDMS) using patterned photoresist such as SU-8. As an alternative material for microfluidic devices, cyclic olefin copolymer (COC) thermoplastics typically provide advantages such as better mechanical robustness as well as less absorption and porosity compared to PDMS, making it a desirable choice for applications in cell culturing including reconstructing and modeling of neural circuit. Microfluidic devices made from COC and other thermoplastics are typically molded using master molds made from metals for robustness and reliability compared to photoresist and silicon molds. However, the fabrication of the metal master mold is challenging, particularly when high aspect ratio and high-resolution features are needed.

Therefore, a need still exists for new microfluidic devices that can provide accurate modeling of neural circuits, as well as methods for integrating microfluidic components in 3D and making highly detailed master molds.

### SUMMARY OF THE INVENTION

The present invention addresses this need with novel designs of the cell culturing and modeling device for both 2D and 3D configurations that are enabled by a robust fabrication and integration process to realize compartmentalized microfluidic devices with high aspect ratio and high-resolution microfluidic features. One embodiment of the present invention is compartmentalized microfluidic device comprising a rigid, transparent substrate; a frame bonded to the substrate, where the frame forms a majority of perimeters for at least two compartments, with openings located between adjoining compartments; and one or more separators. Each separator has a plurality of microfluidic channels and a separator is located in each opening in the frame and each separator is integrated to the frame. In addition, each separator is oriented in a perpendicular direction to the substrate. In one embodiment, the perimeters for the at least two compartments are formed by the frame except for the openings. In another embodiment, the device has two or three compartments. In one embodiment, the substrate comprises glass. In another embodiment, the frame comprises a material selected from the group consisting of polymers, ceramics, metals and glass. In one embodiment, the frame comprises a material selected from the group consisting of thermoplastic polymers and copolymers. In another embodiment, the separator comprises a material selected from the group consisting of polymers, ceramics, metals and glass. In one embodiment, the separator comprises a material selected from the group consisting of thermoplastic polymers and copolymers, PDMS and cyclic olefin copolymer.

In another embodiment, the compartmentalized microfluidic device also includes one or more electrodes. In one embodiment, the electrodes are selected from the group consisting of microelectrode arrays (MEAs) and three-dimensional electrodes. In another embodiment, the electrodes are aligned with the microfluidic channels of a separator. In one embodiment, the electrodes comprise three-dimensional electrodes and one or more of the three-dimensional electrodes are mounted on the substrate, the separator, or both. In another embodiment, each separator has one or more microfluidic channels with at least one dimension smaller than about 10 microns.

In one embodiment, the separator is fabricated from a mold and the mold comprises a material selected from the group consisting of metals, ceramics, silicon, silica, and polymers with a high heat tolerance. In another embodiment, the mold has multiple mold cavities for batch processing.

In one embodiment, the present invention is a method to vertically integrate microfluidic components to realize 3D device features with high aspect ratio (height vs. width).

In one embodiment, the present invention is a process for realizing a compartmentalized microfluidic device with high aspect ratio and high-resolution microfluidic features. The process involves constructing a mold with one or multiple mold cavities; fabricating one or more separators comprising a plurality of microfluidic channels by placing material in the mold and heating; forming a frame; bonding the frame to a rigid transparent substrate, wherein the frame forms a majority of perimeters for at least two compartments, with openings located between adjoining compartments; and placing a separator in each opening in the frame and bonding the separator to the frame. The separator is oriented in a perpendicular direction to the glass substrate. In another embodiment, the mold has releasing holes, wherein pressure is applied through the releasing holes to release the molded separator from the mold.

In one embodiment, the present invention is a method of detecting neural communications between neural tissues or organ tissues. The method involves culturing neural cells, brain organoids, brain tissue, other organ tissue or both in a compartmentalized microfluidic device and detecting neural activity between compartments using electrodes. The device comprises a rigid, transparent substrate; a frame bonded to the substrate, wherein the frame forms a majority of perimeters for at least two compartments, with openings located between adjoining compartments; and one or more separators. Each separator has a plurality of microfluidic channels. Also, a separator is located in each opening in the frame and each separator is bonded to the frame. In addition, each separator is oriented in a perpendicular direction to the substrate. In another embodiment, one or more of the electrodes are 3D electrodes. In one embodiment, the other organ tissue is selected from the group consisting of muscle tissue, heart tissue and organoids.

### BRIEF DESCRIPTION OF THE DRAWINGS

The foregoing summary, as well as the following detailed description of preferred embodiments of the application, will be better understood when read in conjunction with the appended drawings.

FIGS. 1A-1E are illustrations of microfluidic devices for neural circuitry modeling according to the present invention. FIG. 1A shows a first embodiment of the present invention;

FIG. 1B shows a second embodiment of the present invention, FIG. 1C shows a zoom-in view showing details of microchannels, FIG. 1D shows a cross-sectional view, and FIG. 1E shows an example of a 3D configuration of microfluidic channels with vertical integration of microfluidic components.

FIGS. 2A-2H are illustrations showing an example implementation of the process flow for fabrication of a separator with microchannel features and integration of the overall device. FIG. 2A is an illustration of a top-down view of a metal mold fabricated using μEDM. FIG. 2B is an illustration of a cross-sectional view of the metal mold. FIG. 2C is an illustration of a top-down view of a separator being fabricated in the metal mold. FIG. 2D is an illustration of a cross-sectional view of a separator being fabricated in the metal mold. FIG. 2E is an illustration of a top-down view of a separator being released the metal mold. FIG. 2F is an illustration of a cross-sectional view of the molded separator. FIG. 2G is an illustration of a top-down view of a separator being inserted into the frame. FIG. 2H is an illustration of a cross-sectional view of a separator inserted into the frame on top of a substrate.

FIG. 3 is a series of illustrations showing aspects of the mold preparation. FIG. 3A is an illustration of a fine tip of tungsten microtool; FIG. 3B shows a 2×2 array of mold cavities. FIGS. 3C and 3D are illustrations of the micron-scale features on the mold for forming microchannels. FIG. 3C shows top-down view, and FIG. 3D shows perspective view.

FIG. 4 is a schematic of a precision assembly tool for integration of device components. Intimate contact between separator and glass substrate formed by thermal compression without using adhesive.

FIG. 5 is an illustration of a fully assembled device according to the present invention.

FIG. 6 is an example design of microchannels on a separator and their alignment with an 8×8 electrode array on a typical commercial MEA chip.

FIG. 7A is an illustration of an example design of additional row(s) of microchannels on the separator for 3D configuration, showing cross shape for the cross-sections of the channels. FIG. 7B shows example options of cross-sectional shapes of microchannels, including but not limited to cross, rotated cross, triangular arch, rectangle, rounded rectangle, square, circle, triangle, hexagon, and arc.

FIG. 8 is an illustration showing features on the metal mold for making 15 channels on a separator, each channel of 10 μm width and >200 μm length.

FIG. 9 is an illustration showing microchannels on molded COC separator, each channel of 10 μm width and >200 μm length.

FIG. 10 is an illustration showing a COC separator with 15 microchannels bonded to a glass substrate; photo taken through the glass showing uniform and intimate bond between the separator and glass without the use of adhesive.

FIG. 11A is an illustration showing the measured water contact angles on untreated COC surface, which is hydrophobic.

FIG. 11B is an illustration showing the measured water contact angles on a COC surface treated with P100 hydrophilic coating.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

As used herein, the term “three-dimensional electrodes” or “3D electrodes” means electrodes with height or thickness in the direction perpendicular to the substrate larger than the widths or lateral dimensions (parallel to the substrate). Example typical height or thickness can be a few tens of microns to a few millimeters. This is also in contrast to the thin film electrodes that typically have thickness of a few microns or less.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention involves novel designs of compartmentalized microfluidic devices that can be used for neurobiological studies and other bioelectronic measurements during cell culturing or tissue growth. In some embodiments, the devices can be integrated with electrodes, including three-dimensional (3D) electrodes, commercial or custom-made micro-electrode array (MEA) chips to assist in these studies. Unlike previous microfluidic devices that use planar configurations to implement device features in a two-dimensional (2D) horizonal arrangement (i.e. parallel to the substrate surface), the present invention uses a vertical configuration (i.e. perpendicular to the substrate surface) that enables 3-dimensional (3D) channel placement, allowing the study of neural communications between not only neurons, but larger volume neural tissues or even brain organoids. Two or more compartments in the designs allow the study of neuron-neuron connections between multiple neuron species, or even neuronal projections between neurons and tissue from other types of organs such as muscle, heart, etc., thus establishing multiple-organs-on-a-chip.

The present invention discloses novel vertical configuration (perpendicular to the substrate surface) of high aspect ratio microfluidic components with high resolutions. This allows the potential for 3-dimensional (3D) configurations of microfluidic features for modeling larger neural tissues such as organoids as well as other cells, tissue, or organoid types. One major application that is enabled by the devices of the present invention is modeling and study of neural circuits, which can provide better understanding of brain functions and causes of neurological disorders such as Schizophrenia (SCZ), Autism Spectrum Disorder (ASD), Attention-Deficit Hyperactivity Disorder (ADHD), Huntington's disease (HD) and Parkinson disease (PD), leading to potentially improved therapies.

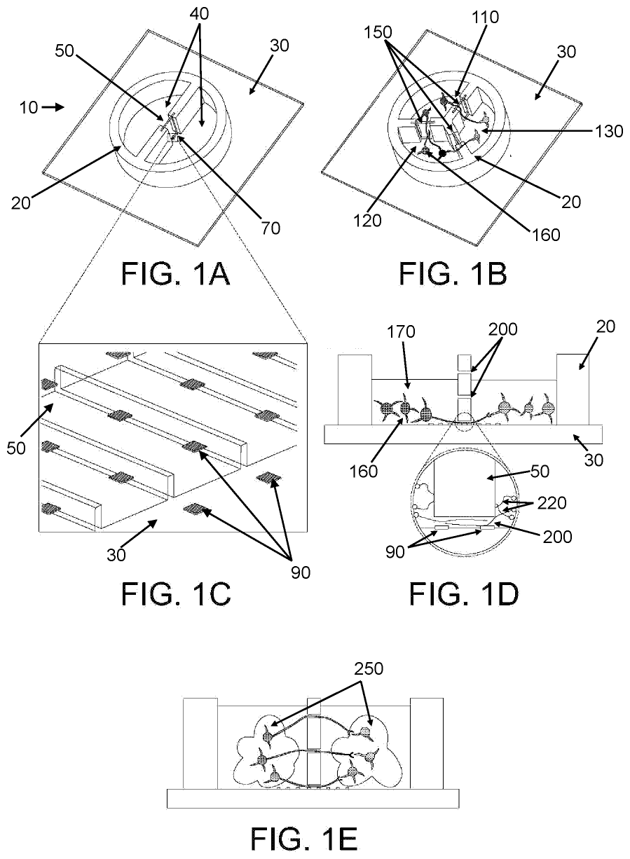

FIG. 1A illustrates one embodiment of the microfluidic device 10 for neural circuit modeling as an example of implementation according to the present invention. In this embodiment, a frame 20 mounted on a transparent substrate 30 forms the majority of the perimeters for two culturing compartments 40, leaving a narrow opening at the center. No microfluidic features are included on the frame. A separator 50 with a group of microfluidic features is inserted into the center opening and bonded to the frame. This completes the division of the two culturing compartments 40, allowing for establishing biologically long-range connections between two neuronal cultures. In this embodiment, the substrate 30 includes an electrode array 70.

FIG. 1B illustrates another embodiment of the present invention. In this embodiment, a frame 20 mounted on a transparent substrate 30 forms the majority of the perimeters for three culturing compartments: a cortical unit 110, a striatal unit 120 and a midbrain unit 130. Three separators 150 with microfluidic features are inserted into the center openings between the culturing compartments and bonded to the frame. The culturing compartments contain neurons 160. Referring to FIG. 1D, a cross-sectional view of the microfluidic device of the present invention is shown. In this embodiment, a frame 20 mounted on a substrate 30 defines culturing compartments that hold culturing fluid 170 and neurons 160. A separator 50 has multiple microchannels 200. Each microchannel 200 is small enough to prevent neurons from migrating through the channels while allowing fluid and axons 220 to transport across the separator 50 and establish axonal connections. Electrodes 90 may be used to collect information.

In FIG. 1E, a cross-sectional view of the microfluidic device of the present invention is shown. In this embodiment, organoids 250 make multiple axonal connections through the vertically arranged microchannels of the separator.

The substrate used in the present invention is rigid and transparent. Various materials can be used to make the substrate, including transparent polymers and glass. In one embodiment, the substrate is made of glass. As shown in FIG. 1C, the substrate 30 may include an electrode array 70, comprised of individual electrodes 90.

The frame can be constructed of a biocompatible material that is mostly impervious to liquid. The frame can be designed with multiple compartments. In one embodiment, the frame has 2, 3 or 4 compartments. In another embodiment, the frame has 2 or 3 compartments. In one embodiment, the frame, which has no microfluidic features, is fabricated from cyclic olefin copolymer sheets (TOPAS® 8007 COC) using a 5-axis CNC micromilling machine for fast prototyping. Injection molding can also be used. Other types of thermoplastic materials can also be used.

In some embodiments, the microfluidic devices of the present invention can be integrated with MEA chips that are commercially available or custom-made for built-in monitoring of neural activities during neuron culturing or for monitoring of bioelectronic signals from other types of tissue. In other embodiments, the microfluidic devices of the present invention can be integrated with 3D electrodes. Since 3D electrodes can penetrate deeper into the tissue and/or organoid, they can measure biological/neuron activity in three dimensions. Therefore, the devices of the present invention provide unique advantages for studying brain organoids and neurons.

The separator is designed to be mounted in a vertical orientation with respect to the culturing compartments of the device of the present invention. The vertical integration of the microfluidic components enables realization of 3D device features with high aspect ratio (height vs. width). When installed, the separator will have a greater height than width. In some embodiments, the separator has a height that is at least 3 times greater than the width of the separator.

In one embodiment, the microchannels on the separator are designed with a size of ≈10 μm (W)×30 μm (H)×200 μm (L), with at least one dimension smaller than the size of human neurons (typically >10 μm) to prevent neurons from migrating through the channels while allowing fluid and axon to transport across the separator and establish axonal connections. Other smaller or larger sizes and different cross-sectional geometries of the channels can be readily realized by changing the process parameters for micromachining. In some embodiments, the microchannels on the separator have at least one dimension smaller than a few microns. In one embodiment, the microchannels on the separator have at least one dimension smaller than about 10 microns.

In another embodiment, the separator, when installed in the opening of the frame, is at least about 1 mm tall. In one embodiment, the separator, when installed in the opening of the frame, is from about 1 mm to about 5 mm tall.

In one embodiment, the fabrication process to create a mold for separators involves the use of an improved micro electro-discharge machining (μEDM) technique. This technique results in novel high levels of aspect ratio and resolution to make molding masters, while other techniques such as micromilling, laser micromachining, and high-resolution 3D printing may also be used to make masters with various resolution and aspect ratios. The mold may be formed from various materials, including metals, ceramics, silicon, silica, and other polymers with high heat tolerance.

In one embodiment, the mold has multiple mold cavities formed using one or more techniques selected from the group consisting of micro electrical-discharge machining (EDM), micromilling, laser micromachining, high-resolution 3D printing, wet etching, plasma etching, ultrasonic and molding.

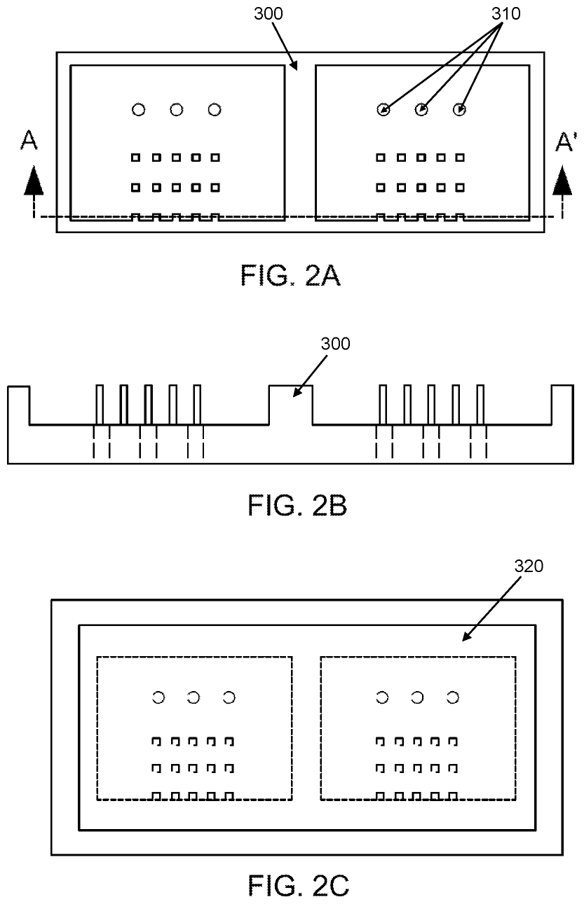

In another embodiment of the process for making the separator, FIGS. 2A-2H illustrate the process flow for its fabrication in the batch mode. A metal mold 300 with multiple mold cavities is first made by μEDM using a Micro-EDM machine (FIGS. 2A and 2B). Directional indicators A-A′ show the direction of the cross-sectional view for FIG. 2B. The metal used for demonstration is brass, while other metal alloys such as stainless steels and titanium alloys can also be used. As one method to achieve the high resolution and high aspect ratio features, the μEDM process and parameters were used (Table 1). A multistep machining with a different tool diameter in each step is implemented. A Φ30 μm tool is used to raster scan the majority of the cavity area. A Φ30 μm tool is used to define the overall shape of the microchannels, which is then finished using a Φ15 μm tool to achieve the target feature sizes. As listed in Table 1, during the machining with Φ15 μm and Φ30 μm tools, the discharge parameters are set to the machine parasitic capacitance level (≈15 pF) and a low discharge voltage at 64 V to obtain small feature sizes and minimum discharge gap along with smooth surface finish. The corresponding discharge energy is as small as 30 nJ.

TABLE 1
Parameters used in μEDM of molds

| | | |
|---|---|---|
| Tool diameter (μm) | 300 | 30/15 |
| Discharge capacitance (pF) | 235 | ≈15 |
| Discharge voltage (V) | 90 | 64 |
| Scanning feed rate (mm/min) | 6 | 1 |
| Vertical feed rate (mm/min) | 0.5 | 0.1 |
| Discharge energy (nJ) | 951.75 | 30.72 |
| Tool wear ratio* | 1.2% | 0.4% |

*Percent of tool length worn by machining depth

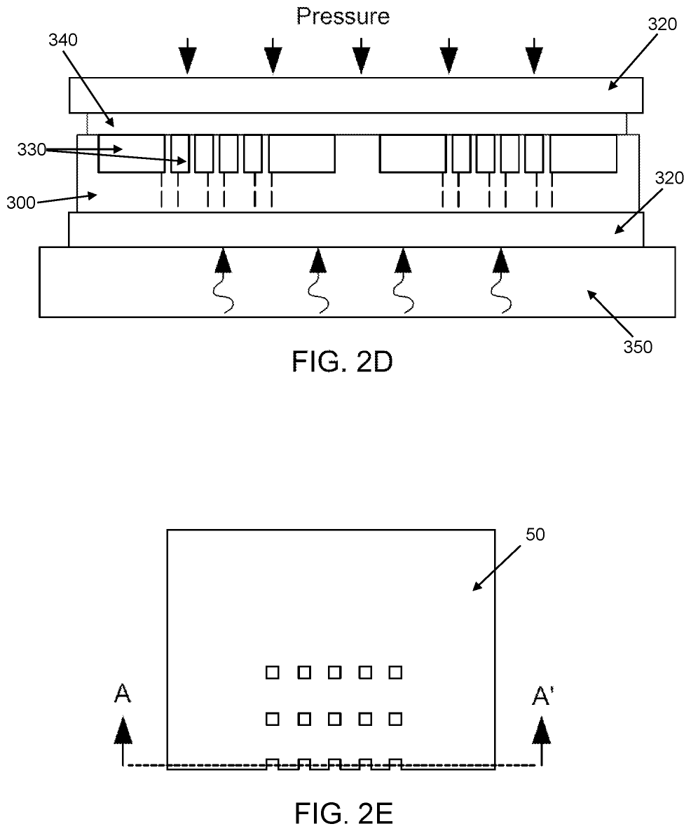

In one embodiment, the microtool 500 for μEDM of the mold is prepared using the wire electro-discharge grinding (WEDG) function of the Micro-EDM machine. A tool length of 250 μm is selected to allow a cutting depth of at least 200 μm for the mold cavity, resulting in an aspect ratio of 16:1 for the 15 μm tool. To minimize wobbling and errors for such large aspect ratio tool, a 30 μm feature at the base of the tooltip is included to form a cone structure, as shown in FIG. 3A.

One of the fabricated molds with a 2×2 array of mold cavities 520 is shown in FIG. 3B, with micron-scale features for forming microchannels shown in FIGS. 3C and 3D. The fabrication technique for the mold is proven to be robust and repeatable, consistently generating features at ≈10 μm width and ≈200 μm height, with an aspect ratio of 20:1. The mold cavities include release holes 530.

Using the fabricated molds, separators are then made by molding as shown in FIGS. 2C and 2D. The mold 300 is sandwiched between two glass substrates 320 after adding TOPAS® 8007 COC into the mold cavities 330 with the desired COC amount controlled by mass. A layer of silicone sheet 340 is attached between the top glass substrate and the mold to form a good seal of the opening of the mold cavity. A pressure of ≈200 kPa is applied to the top glass substrate and the whole sandwiched assembly is placed on a hotplate 350 for the molding process. A temperature of 150° C. is used to fully melt the COC for molding although the glass transition temperature of the material is only 78° C. After molding, the separator 50 is released from the mold by applying pressure through the releasing holes 310 (FIGS. 2E and 2F). The molds have been used repeatedly for many (>20) iterations without significant degradation or damage, indicating the robustness of the mold and the process. Directional indicators A-A′ in FIG. 2E show the direction of the cross-sectional view for FIG. 2F. In another embodiment, the molding process can be implemented with injection molding, hot embossing or other molding techniques and tools using the prepared molds.

Finally, the separator 50 is inserted in the opening of the frame 20 (see FIGS. 2G and 2H). To prepare the compartmentalized microfluidic devices of the present invention, one embodiment bonds the frame to a glass substrate 30 with a biocompatible adhesive 400 such as silicone. The separator is then integrated with the frame using adhesive after its insertion into the slots on the frame; however, no adhesive is used on the interface 410 between the separator and the glass substrate given the small feature sizes of the channels that can be easily blocked by even a thin layer of adhesive. Directional indicators A-A′ in FIG. 2G show the direction of the cross-sectional view for FIG. 2H.

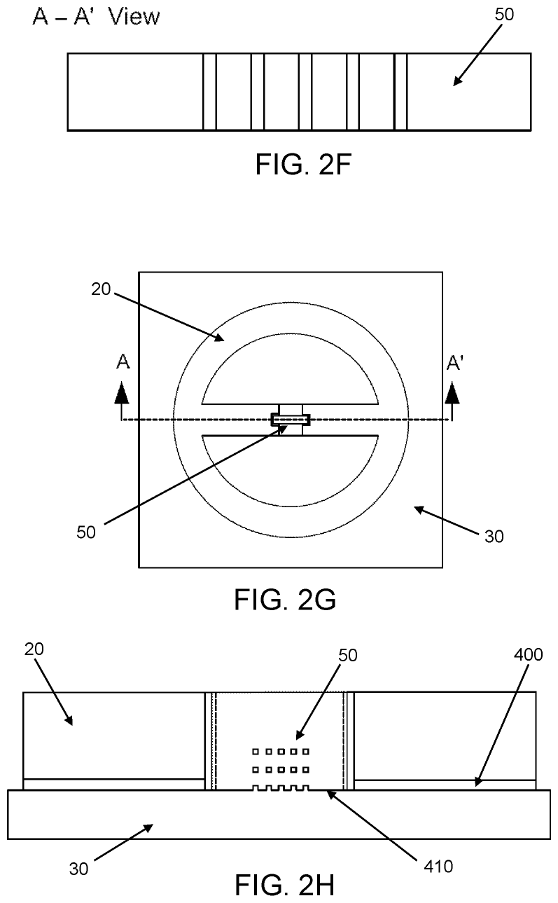

In this embodiment, an intimate contact between the separator 50 and the glass substrate 30 is formed by thermal compression, without any adhesive, using a precision assembly tool shown in FIG. 4. In this tool, the separator 50 is clamped (via clamp 550) to the Z stage 560, which is controlled by a micrometer with submicron resolution, is used to lower the separator 50 into the frame 20 opening and also to apply a pressure while the glass substrate is heated on a hot plate 590 to a temperature of 70° C. The pressure allows the softened bottom surface of the COC separator to be mated to the glass substrate, thus forming an intimate and uniform interface. The tool also includes a precision XY stage with rotational platform 570 as well as a microscope 580, allowing alignment and monitoring of the thermal compression process.

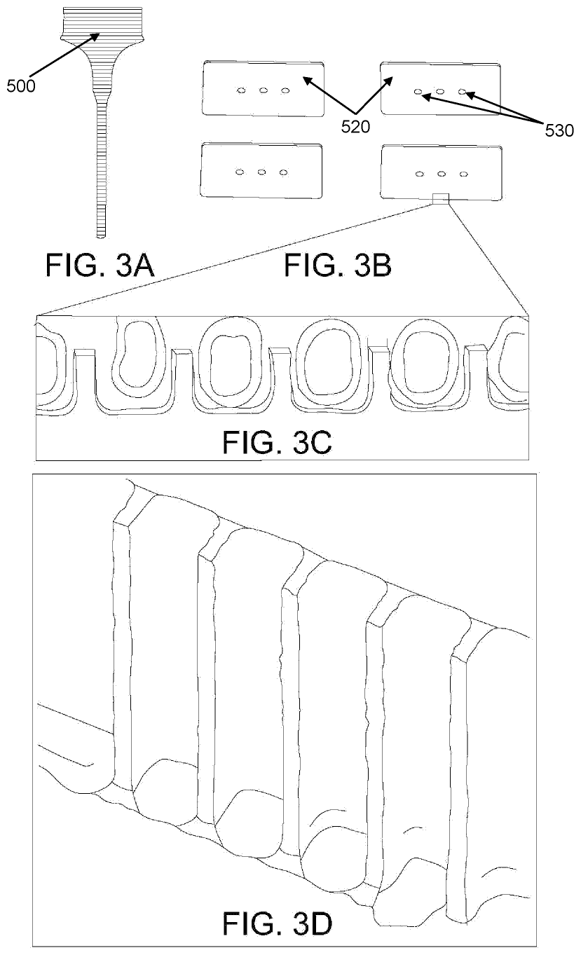

An embodiment of a fully assembled device with the frame 20 and separator 50 integrated on a glass substrate 30 is shown in FIG. 5. There is an intimate and uniform contact interface between the separator and the glass substrate. This interface is formed by thermal compression without the use of an adhesive.

For integration of the microfluidic devices and MEA chips to form microfluidic MEA chips (FIG. 1A, 1C, 1D), MEA chips either commercially available or custom made can be used. These chips have an array of planar electrodes for neuron signal recording. The integration of the multi-compartment microfluidic devices with MEA chips will enable monitoring of neural network dynamics during human brain circuitry development, illustrated as Embodiment 1 in FIG. 1A. For example, human cortico-limbic circuits can be reconstructed on a microfluidic MEAs chip by growing cortical and striatal cultures in their corresponding compartments. The neuronal spike activity and network dynamics can be assessed during human brain circuitry maturation. The neuronal propagation can be also detected by electrodes aligned with microfluidic channels.

Microfluidic devices with two or more independent compartments can be developed to accommodate multiple types of neuron cells or tissues, e.g. cortical, striatal and midbrain neurons (FIGS. 1B, 1C, 1D). These compartments can be interconnected by microfluidic channels with high resolutions as small as a few microns built into the separators, only allowing, e.g. axonal growth between the neurons to reconstruct long-distance neuron communication pathways. This microfluidic devices with two or more independent compartments can be used to reconstruct complex brain circuits such as meso-cortico-limbic circuits by culturing cortical, midbrain and striatal neurons in corresponding unit (shown as Embodiment 2 in FIG. 1). Neuronal projections between neurons and tissue from other types of organs such as muscle, heart, etc. can also be reconstituted by culturing both brain tissue and other organ tissue in separate compartments, thus establishing multiple-organs-on-a-chip. Custom made MEA chip with multiple electrode arrays matched to the design with the multiple chambers can be prepared for integration and monitoring of neural activity.

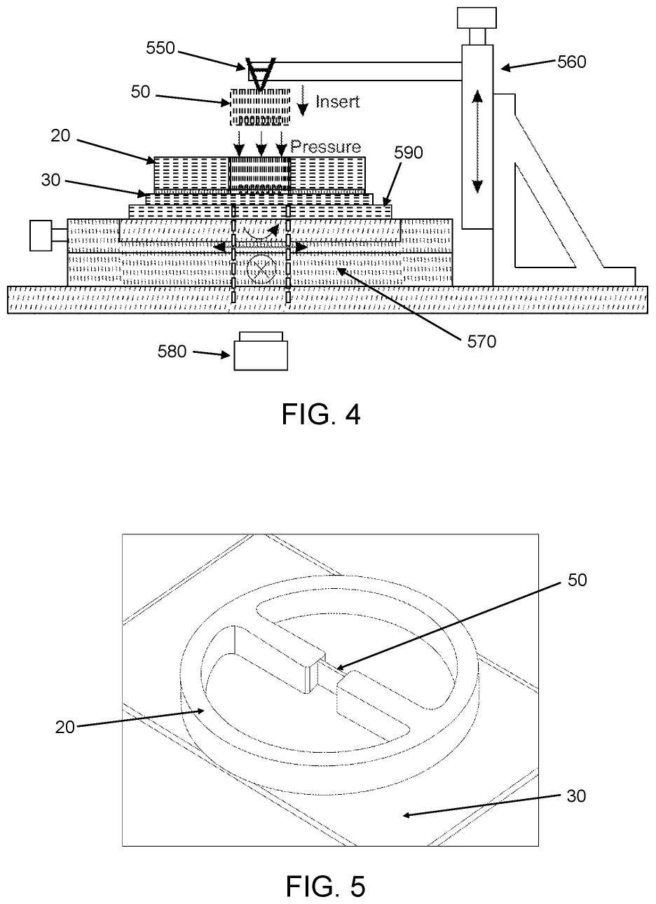

The microchannels on the separator can be custom designed to match the electrode array on the micro electrode array (MEA) chip. Parameters that can be customized include the number, width (W), length (L), and spacing (d_{c}) of the microchannels. FIG. 6 shows an example design with 15 microchannels 600 on a separator 50 to match an 8×8 array of electrodes on a typical commercial MEA chip. Among these 15 microchannels, 8 are aligned with the 8 rows of the electrodes 90 to allow real-time monitoring of neural activities inside the channels, and the others are located in between the electrodes to help increase fluid paths between the culturing compartments on the left and right sides of the separator. Additional microchannels can be added between the electrodes as necessary if space allows. The length L of the microchannels determines the number of electrodes aligned with a single channel; two electrodes are shown for demonstration while more can be added.

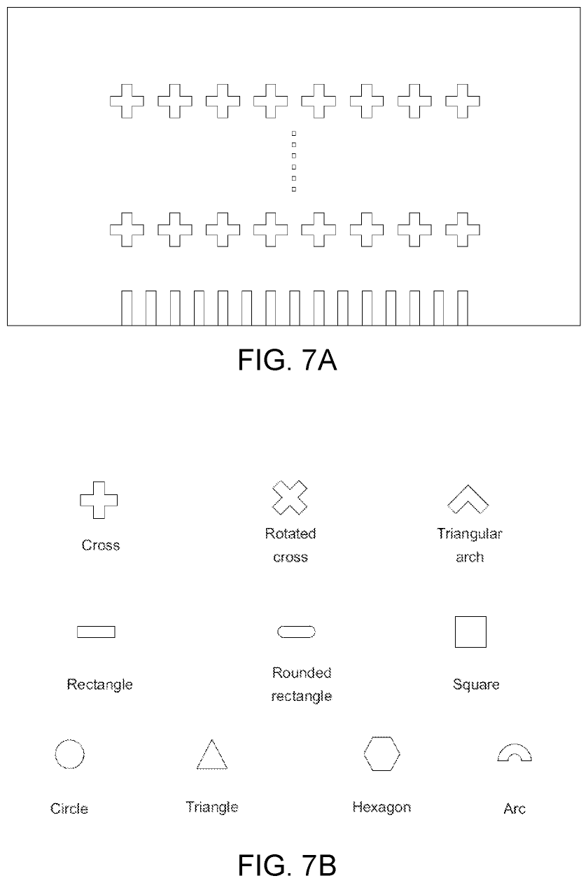

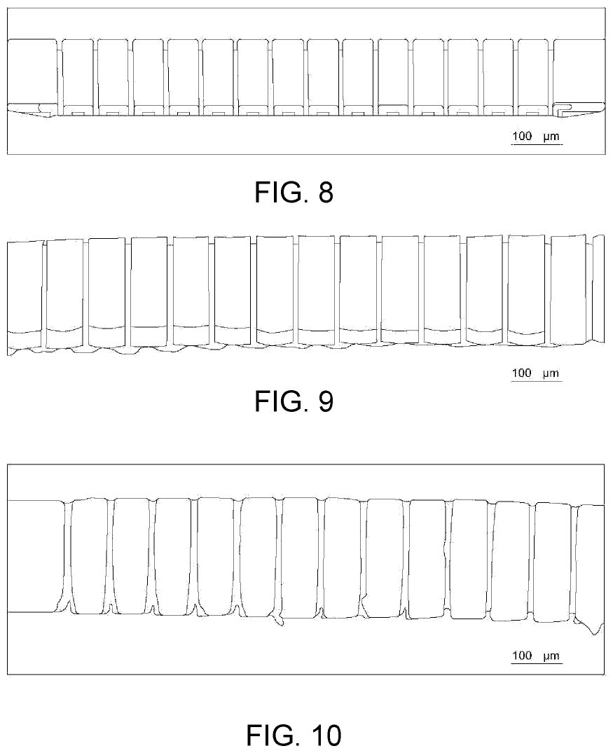

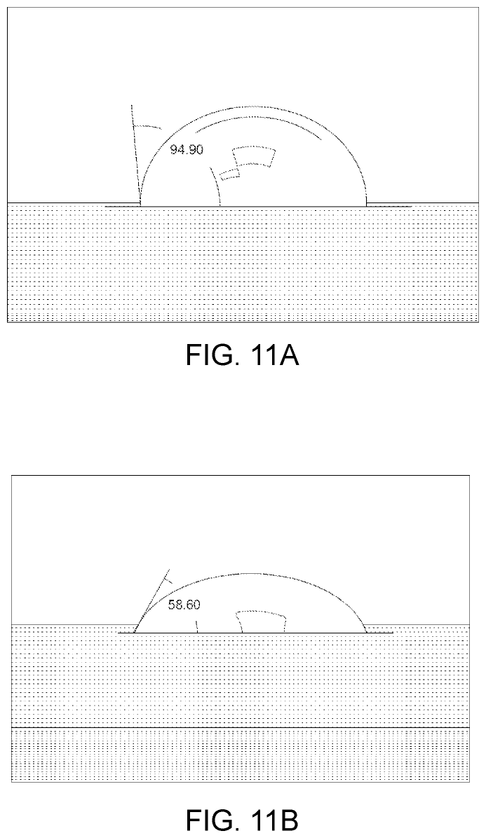

As a demonstration, 15-channel separators have been designed, successfully fabricated, and made ready for integration with a commercial MEA chip (like the one illustrated in FIG. 6). FIGS. 8-10 show, respectively, the features on a metal mold for making separators with 15 channels, each of 10 μm width and >200 μm length; microchannels on the molded COC separator using the mold; and the uniform and intimate bond interface between the 15-channel separator and a glass substrate without the use of adhesive.

The precision alignment tool with microscope observation and micrometer motion control, which was described in FIG. 4, can be used to align the microchannels on a separator to the electrode array on a MEA chip. The electrodes that are aligned with the microchannels are exposed to the axons that grow along the channels between the neurons on the two sides of the separator, allowing real-time monitoring of the neural signals that are transmitted through the axons. Having more than one electrode aligned with a single channel provides better reliability in signal acquisition and can potentially allow tracking of the direction and sequence of the neural signals.

The present approach for vertical integration of microfluidic components can enable built-in microchannels in 3D configurations (FIG. 1E), thus allowing, e.g., reconstruction of 3D brain circuits of multiple brain organoid tissue types within a single microfluidic device, potentially providing greater mechanistic understanding of higher-order brain function such as EEG-like activity with the increased resemblance to in vivo conditions.

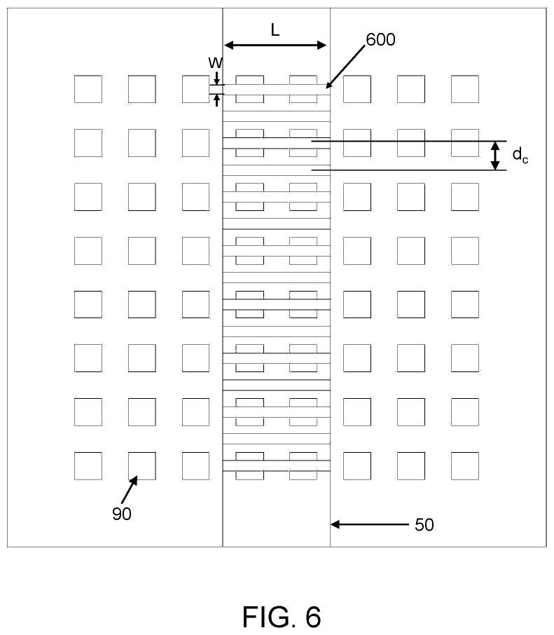

For the design variation with 3D channel configuration, additional rows of microchannels can be added on the separator along the height, away from the bottom substrate of the MEA chip, allowing the use of the device with e.g., organoids. FIG. 7A shows example designs of the additional microchannels and FIG. 7B shows example options for the cross-sectional shape of the microchannels. The number and cross-sectional shapes of the additional channels can be customized with considerations of factors such as structural integrity of the channels as well as feasibility and robustness of the microfabrication process. Electrodes, either thin film or 3D, can also be added on the separator around the openings of the microchannels.

The COC polymer used in some embodiments for molding separators is known to be mildly hydrophobic, which makes it difficult for the culturing fluid to flow between the compartments. Air bubbles can also be more easily trapped in the microchannels when the channel surface is hydrophobic, preventing fluid path from being established between the compartments and axons from growing along the channels.

Options to convert the COC surface from hydrophobic to hydrophilic include surface oxidization, either with strong acid or oxygen plasma, and hydrophilic coating. Either option is feasible for the microfluidic device. The second option was tested using a hydrophilic coating solution such as P100 from Jonsman Innovation ApS, Denmark. An example protocol for the application of a coating solution before neuron culturing is:
1) Dilute P100 with isopropyl alcohol at 1:10 ratio
2) Dip coating the molded separator and proceed with device assembly
3) Sterilize assembled device with ethanol alcohol
4) Rinse device with diluted coating solution again as needed

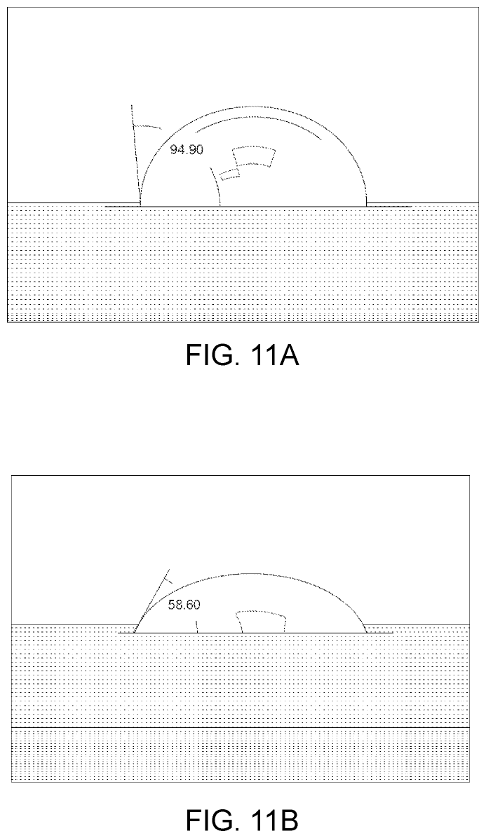

FIG. 11A shows the measured water contact angles on untreated COC surface, which is hydrophobic, and COC surface treated with P100 hydrophilic coating (FIG. 11B). Flow testing indicated that the devices with the hydrophilic coating allow water to freely flow through the microchannels, as opposite to what happens with uncoated devices.

### EXAMPLES

### Example 1

The assembled devices according to the present invention were used in experiments for neuron cell culturing. The device was first sterilized by ultraviolet (UV) for 30 min. before cell culturing. After sterilization, the compartment surfaces of the device were coated by Poly-D-Lysine (PDL) followed with Matrigel, each overnight, for better survival and attachment of neurons to the substrate. Human neural progenitors derived from a human embryonic stem cell (hESC)-H9 were differentiated by neural progenitor markers, e.g. Pax6 and Sox2, and were then seeded into one of the compartments for culturing at 37° C. and 95% humidity in neuronal culture medium consisting of neurobasal medium supplemented with 2 mM L-glutamine, B27, 10 ng/ml BDNF (PeproTech), 10 ng/ml GDNF (PeproTech), 200 ng/ml Ascorbic Acid (AA, Sigma) and 1 μM cAMP (Sigma).

After a 2 week period of culturing, human neurons became mature in the device and started to extend neurites including axons. To label the neurites in the microchannels, green fluorescent protein (GFP) was used. Fluorescent images were taken through the glass substrate using a Nikon upright microscope Eclipse Ni-E. The images showed that neurites had entered all five microchannels, providing preliminary verification of the validity of the design and functions of the device for neuron culturing and circuit modeling.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A compartmentalized microfluidic device comprising:
a. a rigid, transparent substrate;
b. a frame bonded to the substrate, wherein the frame forms a majority of perimeters for at least two compartments, with openings located between adjoining compartments; and
c. one or more separators, each separator comprising a plurality of microfluidic channels, wherein a separator is located in each opening in the frame and each separator is bonded to the frame;
wherein each separator is oriented in a perpendicular direction to the substrate.

2. The compartmentalized microfluidic device of claim 1 wherein the perimeters for the at least two compartments are formed by the frame except for the openings.

3. The compartmentalized microfluidic device of claim 1 wherein the device has two or three compartments.

4. The compartmentalized microfluidic device of claim 1 wherein the substrate comprises glass.

5. The compartmentalized microfluidic device of claim 1 wherein the frame comprises a material selected from the group consisting of polymers, ceramics, metals and glass.

6. The compartmentalized microfluidic device of claim 1 wherein the frame comprises a material selected from the group consisting of thermoplastic polymers and copolymers.

7. The compartmentalized microfluidic device of claim 1 wherein the separator comprises a material selected from the group consisting of polymers, ceramics, metals and glass.

8. The compartmentalized microfluidic device of claim 1 wherein the separator comprises a material selected from the group consisting of thermoplastic polymers and copolymers, PDMS and cyclic olefin copolymer.

9. The compartmentalized microfluidic device of claim 1 further comprising one or more electrodes.

10. The compartmentalized microfluidic device of claim 9 wherein the one or more electrodes are selected from the group consisting of microelectrode arrays (MEAs) and three-dimensional electrodes.

11. The compartmentalized microfluidic device of claim 9 wherein one or more of the electrodes are aligned with the microfluidic channels of a separator.

12. The compartmentalized microfluidic device of claim 9 wherein one or more of the electrodes comprise three-dimensional electrodes and wherein one or more of the three-dimensional electrodes are mounted on the substrate, the frame, or both.

13. The compartmentalized microfluidic device of claim 1 wherein each separator has one or more microfluidic channels with at least one dimension smaller than about 10 microns.

14. The compartmentalized microfluidic device of claim 1 wherein the separator is formed from a mold and the mold comprises a material selected from the group consisting of metals, ceramics, silicon, silica, and polymers with a high heat tolerance.

15. The compartmentalized microfluidic device of claim 14 wherein the mold has multiple mold cavities.

16. A process for fabricating a compartmentalized microfluidic device with high aspect ratio and high-resolution microfluidic features comprising:
a. constructing a mold with one or multiple mold cavities;
b. forming one or more separators comprising a plurality of microfluidic channels by placing material in the mold and heating;
c. forming a frame;
d. bonding the frame to a rigid transparent substrate, wherein the frame forms a majority of perimeters for at least two compartments, with openings located between adjoining compartments; and
e. placing a separator in each opening in the frame and bonding the separator to the frame;
wherein the separator is oriented in a perpendicular direction to the glass substrate.

17. The process of claim 16 wherein the mold has releasing holes, wherein pressure is applied through the releasing holes to release the molded separator from the mold.

18. A method of detecting neural communications between neural cells or brain organoids comprising culturing brain tissue, other organ tissue or both in a compartmentalized microfluidic device and detecting neural activity between compartments using electrodes, the device comprising:
a. a rigid, transparent substrate;
b. a frame bonded to the substrate, wherein the frame forms a majority of perimeters for at least two compartments, with openings located between adjoining compartments; and
c. one or more separators, each separator comprising a plurality of microfluidic channels, wherein a separator is located in each opening in the frame and each separator is bonded to the frame;
wherein each separator is oriented in a perpendicular direction to the substrate.

19. The method of claim 18 wherein one or more of the electrodes are 3D electrodes.

20. The method of claim 18 wherein the other organ tissue is selected from the group consisting of muscle tissue, heart tissue and organoids.
